(19) United States
(12) Patent Application Publication
(10) Pub. No.: US 2026/0069613 A1
(43) Pub. Date: Mar. 12, 2026

(54) CANNABINOID AND TERPENE COMPOSITION TO BE APPLIED ON AN ABSORBENT MATERIAL.

(71) Applicant: TRIBU HEMP LLC, Hialeah Gardens, FL (US)

(72) Inventors: Karina Cros, Hialeah, FL (US); Cristian Camilo Diaz Merchan, Bogota (CO)

(73) Assignee: TRIBU HEMP LLC, Hialeah Gardens, FL (US)

(21) Appl. No.: 19/105,355

(22) PCT Filed: Aug. 22, 2023
(86) PCT No.: PCT/US2023/030754

§ 371 Date: Feb. 21, 2025

Related U.S. Application Data
(60) Provisional application No. 63/400,049, filed on Aug. 23, 2022.

(51) Int. Cl. A61K 31/00; A24B 15/28; A24B 15/30; A24B 15/32; A24B 15/34; A24C 5/00; A24D 1/00; A24D 1/02; A61K 31/015; A61K 31/045; A61K 31/085; A61K 31/22; B23K 26/38; D21H 19/10

(52) U.S. Cl. CPC A61K 31/658; A24B 15/282; A24B 15/301; A24B 15/32; A24B 15/34; A24B 15/345; A24C 5/005; A24D 1/002; A24D 1/02; A61K 31/015; A61K 31/045; A61K 31/085; A61K 31/22; B23K 26/38; D21H 19/10

(57) ABSTRACT

The invention refers to a composition consisting of at least one previously selected and extracted cannabinoid and at least one previously selected and extracted terpene to be applied on a material in order to be absorbed and then released by heat for recreational or medical purposes a paper in which is applied said composition, and a process of manufacturing said paper.

### CROSS-REFERENCE TO RELATED APPLICATIONS

This PCT application claims the benefit of U.S. Provisional Patent Application Ser. No. 63/400,049, filed on Aug. 23, 2022, which is incorporated herein.

### BACKGROUND OF THE INVENTION

### Field of the Invention

The present invention relates to a composition of at least one previously selected and extracted cannabinoid and at least one previously selected and extracted terpene to be applied on a material in order to be absorbed and then released by heat for recreational or medical purposes, also relates about a paper in which is applied said composition, and a process of manufacturing said paper.

### Description of Related Art

Plant-derived compositions, especially cannabis, have rising now accepted recreational uses and rise used to medical treatments.

Therefore, there is a need to generate compositions that can be adapted to each specific use, through the prior selection and extraction of components from vegetable material, which, when mixed, make it possible to obtain a useful and necessary particular composition to satisfy a particular need.

The sensations perceived from the senses contribute to the psycho-emotional well-being of people, so the general objective is to stimulate senses through a composition designed for this purpose, through of components that acts on multiple senses in a combined and simultaneous way.

In such a context, is a problem to obtain a solution that can be useful for recreational and/or medical purposes, by means of a composition based on previously selected and extracted components such as; an at least one previously selected and extracted cannabinoid and at least one previously selected and extracted terpene

In a specific way, the state of the art requires solutions for medical purposes, in particular for problems such as; pain, anxiety, insomnia, low sex drive, individual and group nervousness, acute stress disorder.

In another and complementary way, can obtain an absorbent surface of material that can absorb a composition of at least one previously selected and extracted cannabinoid and at least one previously selected and extracted terpene, that can release the content of said composition by application of heat is another problem for the state of the art.

In a particular sense, it is also a problem of the state of the art to be able to obtain a Rolling paper from paper that can previously be impregnated by spraying with a composition of at least one previously selected and extracted cannabinoid and at least one previously selected and extracted terpene, that can release the content of said composition by application of heat

Determine a process to impregnate by spraying a composition of at least one previously selected and extracted cannabinoid and at least one previously selected and extracted terpene in a paper for use to made rolling papers, pre rolls, or cones, is also a problem of the state of the art.

Other means of administering the composition of at least one previously selected and extracted cannabinoid and at least one previously selected and extracted terpene, would be desirable, in such a way that they can be released into the human or animal body by its internal heat, such as beverage drinks, infusions, transdermal patches, and others means such as; humidifiers, e-cigarettes, scent bars, cones, pre rolls, and vaporizers, are important to solve for the state of the art.

The state-of-the-art reports multiple solutions among which are the following, as the closest to the technology proposed by the present invention.

For example, US 2018 360103 to Kaplan et al, discloses a rolling paper formed according to a modified conventional paper-making process. The end product is infused with cannabinoids from an oil extract from the cannabis plant. Since paper-making is highly water intensive and the cannabinoid containing cannabis extract is an oil that is ordinarily immiscible in the water, an emulsifier is used to allow the cannabinoids to evenly disperse in the making of the paper. The addition of the cannabinoids and the emulsifier is done prior to the drying of the paper. Preferably, the emulsifier and cannabinoids are added during the sizing step of the paper-making. Preferably the emulsifier is a starch-like carbohydrate, a modified starch, more preferably hydroxy-propyl-methyl-cellulose. The cannabinoid containing cannabis extract can be added at the pulping, web-forming step and/or preferably at the sizing step of paper-making.

In another example, US 2022 022525 to Bosh-Sanchez, shows a rolling paper structure and method for making a hand rolled smoking article wherein said rolling paper structure is filled with a filler material and rolled to form a smoking structure such as a cigarette. The rolling paper structure comprises detachable sections and it is configured to create smoking structure with an integrated mouthpiece.

In yet another example, WO 2020 167815 to Rousseau et al, discloses a wrapping material for a smoking article is formed from a web that includes extracted pieces of cannabis combined with web building fibers. The web has a basis weight of from about 20 gsm to about 80 gsm and a permeability of from about 10 Coresta to about 100 Coresta. The wrapping material has a natural cannabis smell and taste. The wrapping material is tobacco and nicotine free and can be used as a carrier for flavorings or aerosol delivery compositions.

None of the known techniques report using a process for applying a composition based on a mixture of at least one cannabinoid and at least one terpene.

And in a specific way, where said composition is applied by spraying on a paper, in order to generate a coating layer on said paper, and when it is used to prepare the corresponding rolling paper for cigarette, pre rolls, or cone, and later light it and inhale it, it can be assimilated by the user, and he experiences a favorable change in his psycho-emotional state and fullness.

Neither do they mention or suggest techniques that techniques to obtain a composition based on at least one cannabinoid and one or more terpenes, which can be applied by spraying on a paper, to create a coating layer on it, where said composition is at least 60% purity cannabidiol and one or more terpenes, in order to have therapeutic effects on mood, such as; analgesics, calming of states of anxiety and insomnia, relaxants for individuals or groups of individuals in physical proximity, disinhibitory of behavior in intimacy, and calming of extreme stress situations.

In this sense, the techniques described above are not oriented in the same sense as what the present invention proposes, so that even when they are in the same technological sector, they could not be taken as interfering with the patentability of what the present invention proposes.

### SUMMARY OF THE INVENTION

The general goal of the present invention is to provide a to a composition of at least one previously selected and extracted cannabinoid and at least one previously selected and extracted terpene to be applied on a material in order to be absorbed and then released by heat for recreational or medical purposes.

Said composition is applied on a material that can be absorbed it and then released by heat for recreational or medical purposes.

This can be used in many forms such as; beverage drinks, infusions, transdermal patches, and others means such as; rolling papers, humidifiers, e-cigarettes, scent bars, dermal creams, body oils, cones, pre rolls, and vaporizers

And, with this composition, the invention seeks as a general objective to improve an emotional experience, obtain good vibrations, an experience of fullness in your emotional state, contributing with therapeutic effects.

Specific goals are proposed to solve specific therapeutic problems such as; pain, anxiety, insomnia, low sex drive, individual and group nervousness, acute stress disorder

Each particular problem requires a specific solution within the context of the proposed invention, each of which is detailed below

One specific goal for the treatment of pain that the invention addresses is a composition consisting of a cannabidiol and at least one antinociceptive terpene.

Pain is an unpleasant sensory and emotional experience associated with, or resembling that associated with, actual or potential tissue damage

Another specific goal of the invention consists of propose a composition of a cannabidiol and at least one terpene selected from; D-limonene, Eugenol, Linalool, Geraniol that activates calming effects for states of anxiety.

Anxiety is an emotion characterized by feelings of tension, worried thoughts, and physical changes like increased blood pressure, also is a disorder involves persistent and excessive worry that interferes with daily activities.

Another specific goal of the invention consists of propose a composition a cannabidiol and at least one terpene selected from; Linalool, β-caryophyllene, D-limonene, Humulene that activates calming effects for states of insomnia

Insomnia is a sleep disorder characterized by difficulty falling asleep, staying asleep, or both.

Another specific goal of the invention consists of propose a composition of a cannabidiol and at least one terpene selected from; α-pinene, β-pinene, Borneol, Linalool, D-Limonene, Geraniol that activates therapeutic effects for increase sex drive

Low sex drive is decreased interest in sexual activity, and sexual thoughts and/or lack of response to sexual stimulation

Another specific goal of the invention consists of propose a composition of a cannabidiol and at least one terpene selected from; Citronellol, Geraniol, Linalool, limonene, α-pinene, Sabinene, that activates therapeutic effects for individual relaxation, generating a state of well-being and relaxation in the short term

Another specific goal of the invention consists of propose a composition of a cannabidiol and at least one terpene selected from; Limonene, Linalyl acetate, Linalool, α-pinene, Borneol, that activates therapeutic effects for group's relaxation, generating a state of well-being and relaxation in the long term

Nervousness is a natural reaction to a stressful situation, many physical sensations can accompany nervousness, including a dry mouth, sweaty palms, and dizziness, may also experience feelings of self-doubt, these feelings and sensations are both uncomfortable and uncontrollable.

Individual nervousness affects a specific person due to intrinsic causes, and is closely related to short-term relaxation.

Group's nervousness is a condition due to interactions of people, and is closely related to long-term relaxation

Another specific goal of the invention consists of propose a composition of a cannabidiol and at least one terpene selected from; α-pinene, Borneol, Linalyl acetate, Linalool, β-pinene that activates therapeutic effects for reduce and controlling acute stress disorder

The means for applying the composition to the absorbent material are varied.

In some cases, the absorbent material is paper, with which Rolling papers, pre-Rolls, and cones can be made.

Rolling paper is a piece of paper used to roll cigarettes, being they tobacco or cannabis

Also, the composition can be used in vaporizers, body oils, dermal creams, beverage drinks, chewing gum, scent bars, bath soap, air fresheners

Cannabinoids according to the invention can be; cannabidiol, tetrahydrocannabinol, Cannabinol, Delta-8-tetrahydrocannabinol, Delta-9-Tetrahydrocannabinol, Delta-10 tetrahydrocannabinol, Tetrahydrocannabiphorol, and Hexahydrocannabinol

Cannabidiol is a compound obtained from the cannabis sativa plant, also known as hemp cannabis, its molecular formula is C₂₁H₃₀O₂

CBD is an abbreviation for cannabidiol.

Cannabidiol is a phytocannabinoid derived from the Cannabis species, which in addition to relieving anxiety, also has other benefits such as; analgesic, anti-inflammatory, antineoplastic and chemopreventive activities.

There is interest in two forms of CBD, CBD isolate or CBD full spectrum.

CBD isolate is a pure form of CBD, that it exclusively contains CBD.

CBD full spectrum contains small amounts of other cannabis compounds.

According with this invention, CBD must be between 60 to 99% purity

Another cannabinoid, such as tetrahydrocannabinol, has been associated with an anxiogenic and generally psychoactive response, on the contrary way, the anxiolytic activity has been attributed mainly to cannabidiol

THC is an abbreviation for tetrahydrocannabinol.

According with this invention the proportion of THC in a composition must be less than 0.3%

Cannabinol is a cannabinoid found in the cannabis sativa plant, and it forms when THC degrades

CBN is an abbreviation for Cannabinol

Delta-8-tetrahydrocannabinol is a psychoactive cannabinoid, and is a mildly euphoric isomer of psychoactive Delta-9 THC

Delta-8 THC is an abbreviation for Delta-8-tetrahydrocannabinol

Another psychoactive cannabinoid is Delta-9 tetrahydrocannabinol.

Delta-9 THC is an abbreviation for Delta-9-tetrahydrocannabinol

According with this invention the proportion of Delta-9 THC in a composition must be preferably less than 0.3%

Another psychoactive cannabinoid is Delta-10 tetrahydrocannabinol

Delta-10 THC is an abbreviation for Delta-10-tetrahydrocannabinol

Tetrahydrocannabiphorol is another psychoactive cannabinoid

THC P is an abbreviation for Tetrahydrocannabiphorol

Hexahydrocannabinol is a hydrogenated derivative of tetrahydrocannabinol

HHC is an abbreviation for Hexahydrocannabinol

Terpenes are aromatic compounds found in plants, including cannabis but not exclusively.

Terpenes can not only be used for aromatherapy but are also approved as flavorings in the food industry, being non-toxic compounds.

The therapeutic properties will depend specifically on the specific terpene

The terpenes most present in the cannabis plant and that form the majority of its essential oil are the monoterpenes myrcene, pinene, limonene, linalool, eucalyptol, and the sesquiterpene caryophyllene.

The variation in the ratio between these terpenes is what produces the wide range of aromas found in the Cannabis plant and now it is beginning to be discovered that they can also participate in the variety of pharmacological effects produced by cannabis and produce synergy with cannabinoids

The problem that arises is to have a means to satisfy therapeutic needs for mood modulation, particularly but not exclusively on pain, anxiety, insomnia, low sex drive, individual and group nervousness, acute stress disorder control.

In essence, it is intended to obtain a means so that the user can, according to their need for an emotional experience, obtain good vibes, an experience of fullness in their emotional state, and therapeutic effects.

In a preferred embodiment the means to obtain as a solution should not generate dependency or anxiety in the user, so isn't contain tetrahydrocannabinol.

A preferred composition based on the mixture of high purity cannabidiol with at least one terpene, allows combining therapeutic effects, especially those related to modulation of moods in a harmonic way, without generating additions or anxiety states.

The present invention proposes to overcome the problems mentioned above, this is possible by preparing a composed mixing from at least 60% purity cannabidiol with at least one terpene, and could be applied by spraying over paper surface, creating a coating surface adhered to said paper, in order to generate a coating layer on said paper, and when it is used to prepare the corresponding cigarette, and later light it and inhale it, it can be assimilated by the user, having experiences a favorable change in his psycho-emotional state and fullness.

Mainly benefits is provide effects on mood, such as; analgesics, calming of states of anxiety and insomnia, relaxants for individuals or groups of individuals in physical proximity, disinhibitory of behavior in intimacy, and calming of extreme stress situations.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or process steps.

Words “preferably” and/or “preferred” means that the declared option is taken as priority, but is not the only one choice, so another option could be used without it being considered that this affects the scope of what is claimed.

This invention discloses a process to spraying on a paper a composition based on cannabidiol and one or more terpenes, to create a coating layer on it, where said composition is at least 60% pure cannabidiol and one or more terpenes, in order to have therapeutic effects on mood.

Other objects, features and advantages of the present invention will become apparent from the detailed description. It should be understood, however, that detailed description, and examples, while indicating specific embodiments of the invention, are given by way of illustration only and are not meant to be limiting.

On the other hand, it is contemplated that changes and modifications within the spirit and scope of the invention will become apparent to those skilled in the art at this detailed description.

### DESCRIPTION OF THE INVENTION

The invention proposes a composition of at least one previously selected and extracted cannabinoid and at least one previously selected and extracted terpene to be applied on a material in order to be absorbed and then released by heat for recreational or medical purposes.

The present invention also provides a solution consisting of a paper that contains adhered to its surface a coating based on a mixed composition of at least one cannabinoid, preferably a CBD, and at least one terpene, such that by combining their effects they allow a specific therapeutic action, among which, depending on the mixture, it can be; analgesics, calmers of states of anxiety and insomnia, relaxants for individuals or groups of individuals in physical proximity, disinhibition of behavior in intimacy, and calmers of situations of extreme stress.

In a preferred embodiment, this invention comprises a paper surface coated by a mixed composition formed by at least 60% CBD and at least one terpene.

The present invention also provides a process to produce a paper that contains adhered to its surface a coating based on a mixed composition of at least one cannabinoid and at least one terpene.

Then, this invention comprises three mainly inventions; a composition, a paper impregned by a composition, and a process to obtain said paper.

### A. Composition

In a general embodiment, this invention comprises a composition of at least one previously selected and extracted cannabinoid and at least one previously selected and extracted terpene to be applied on a material in order to be absorbed and then released by heat for recreational or medical purposes

In a preferred embodiment, this invention comprises a composition of at least one previously selected and extracted cannabinoid and at least one previously selected and extracted terpene to solve specific therapeutic problems such as; pain, anxiety, insomnia, low sex drive, individual and group nervousness, acute stress disorder

In a preferred embodiment, the invention comprises a composition for the treatment of pain, which said composition consisting of a cannabidiol and at least one antinociceptive terpene

In another preferred embodiment, the invention comprises a composition of a cannabidiol and at least one terpene selected from; D-limonene, Eugenol, Linalool, Geraniol for activates calming effects for states of anxiety

In another preferred embodiment, the invention comprises a composition a cannabidiol and at least one terpene selected from; Linalool, β-caryophyllene, D-limonene, Humulene for activates calming effects for states of insomnia

In another preferred embodiment, the invention comprises a composition of a cannabidiol and at least one terpene selected from; α-pinene, β-pinene, Borneol, Linalool, D-Limonene, Geraniol for activates therapeutic effects for increase sex drive

In another preferred embodiment, the invention comprises a composition of a cannabidiol and at least one terpene selected from; Citronellol, Geraniol, Linalool, limonene, α-pinene, Sabinene, for activates therapeutic effects for individual relaxation, generating a state of well-being and relaxation in the short term

In another preferred embodiment, the invention comprises a composition of a cannabidiol and at least one terpene selected from; Limonene, Linalyl acetate, Linalool, α-pinene, Borneol, for activates therapeutic effects for group's relaxation, generating a state of well-being and relaxation in the long term

In another preferred embodiment, the invention comprises a composition of a cannabidiol and at least one terpene selected from; α-pinene, Borneol, Linalyl acetate, Linalool, β-pinene for activates therapeutic effects for reduce and controlling acute stress disorder

In a general embodiment, for each one liter of composition are between 2 to 200 gr of cannabinoid, and between 5 to 100 ml of terpenes

In a preferred embodiment, for each one liter of composition are between 52 to 64 ml of terpenes, and between 36 to 49 gr of cannabidiol.

In a preferred embodiment, the composition can be applied by impregnate to a paper to made any of following products; rolling paper, pre rolls, cones

In another preferred embodiment, the composition can be impregnate by sprayed to paper surface in order to made any of following products; rolling paper, pre rolls, cones

In another preferred embodiment the composition can be applied to made any of following products; vaporizers, body oils, dermal creams, beverage drinks, chewing gum, scent bars, bath soap, air fresheners

Cannabinoids can be used according to the invention are any of following; cannabidiol, tetrahydrocannabinol, Cannabinol, Delta-8-tetrahydrocannabinol, Delta-9-Tetrahydrocannabinol, Delta-10 tetrahydrocannabinol, Tetrahydrocannabiphorol, and Hexahydrocannabinol

### B. Paper Impregned by a Composition

In one general embodiment, this invention comprises a paper wherein its surface is covered by a layer adhered by spraying of a mixture composition of at least one previously selected and extracted cannabinoid and at least one previously selected and extracted terpene, and wherein for each one liter of composition are between 2 to 200 gr of cannabinoid, and between 5 to 100 ml of terpene

In a preferred embodiment of the invention, the least one previously selected and extracted cannabinoid is cannabidiol (CBD)

In another preferred embodiment of the invention, the cannabidiol selected is CBD full spectrum

In another preferred embodiment of the invention, the cannabidiol selected is CBD isolate

In a preferred and alternative embodiment of the invention, the mixed composition to be applied comprising at least of 60% purity CBD and at least one terpene.

The paper according with this invention for use to made rolling papers, pre rolls, or cones

In a preferred and alternative embodiment of the invention, the mixed composition to be applied on paper surface comprising at least 81% CBD purity

In another preferred and alternative embodiment of the invention, the mixed composition to be applied on paper surface comprising between 90% to 99.9% CBD purity

In another preferred and alternative embodiment of the invention, the mixed composition to be applied could comprise more than a one different class of terpene

Each portion of paper is obtained by cutting pieces of a mother sheet of paper, whose dimensions are considerably larger than each portion of paper.

Paper material is vegetal origin selected from; linen, wood, rice or hemp

In a preferred and alternative embodiment of the invention, the paper is from wood.

In a preferred and alternative embodiment of the invention, the paper is from wood, selected from the Fagaceae family, and more particularly of those selected among; Quercus robur, Quercus petraea, Quercus acutissima, Quercus canariensis, Quercus x cerrioide, Quercus cerris, Quercus coccifera, Quercus coccinea, Quercus faginea, Quercus frainetto, Quercus glauca, Quercus hartwissiana, Quercus ilex, Quercus ilex subsp. Ilex, Quercus macrocarpa, Quercus myrsinifolia, Quercus nigra, Quercus phillyreoides, Quercus polymorpha, Quercus pubescens, Quercus pirenaica, Quercus serrata, Quercus shumardii, Quercus suber, Quercus virginiana and Quercus rubra.

Each paper has a grammage of between 9 gr/m² up to 25 gr/m².

Each therapeutic effect obtained by means of the present invention requires a particular combination of CBD with terpenes, as will be detailed below:

Composition of CBD with antinociceptive Terpenes, mixed to increase the pain threshold generating the sensation of relief, and thereby achieve an analgesic effect

Composition of CBD with D limonene, eugenol, linalool and geraniol, mixed in order to generate calming effects in states of anxiety.

Composition of CBD with linalool, β-caryophyllene, D-limonene, and Humulene, mixed in order to generate relaxing effects to control states of insomnia.

Composition of CBD with α-pinene, β-pinene, Borneol, linalool, D-limonene and Geraniol, mixed which seek to increase hormonal schemes for intimacy.

Composition of CBD with terpenes of citronellol, geraniol, linalool, limonene, α-pinene and sabinene, mixed which generate a state of well-being and relaxation in the short term.

Composition of CBD with limonene, linalyl acetate, linalool, α-pinene and borneol, mixed which generate a state of well-being and relaxation in the long term

Composition of CBD with α-pinene, borneol, linalyl acetate, linalool and β-pinene, mixed which generate a state of relaxation and rest for moments of extreme stress.

Each formulation has its own composition and aroma, which generates a different user's experience according to the mix selected.

### C. Process to Apply a Composition on a Paper Surface

The process to apply a composition of at least one previously selected and extracted cannabinoid, preferably a CBD, and at least one previously selected and extracted terpene and wherein for each one liter of composition are between 2 to 200 gr of cannabinoid, and between 5 to 100 ml of terpene, on a paper surface with the corresponding steps as follow:
(a) Supply a paper mother sheet obtained from vegetable, selected from; linen, wood, rice or hemp.
(b) Supply a flat surface with dimensions slightly larger than said paper mother sheet.
(c) Fix paper mother sheet corners to said flat surface.
(d) Supply a mix composition of CBD with at least one terpene.
(e) Supply a container that can be sealed in which to store the previous mix composition
(f) Supply spraying medium to apply the previous mixture on the surface of the paper mother sheet, where said spray means are located in superior proximity with respect to said sheet of paper and can be connected by sealed union with said container
(g) Supply a holding and travel mechanism to hold the spraying medium, wherein said mechanism allows an automated two-dimensional path of said spraying medium, parallel and over proximity to paper mother sheet surface.
(h) Supply a programmable laser cutter mechanism.
(i) Prepare CBD and at least one terpene mix composition.
(j) Pour a portion of said mix composition into the container and seal.
(k) Connect the container with the spraying medium.
(l) Hold the spraying medium with the holding and travel mechanism.
(m) Program the holding and travel mechanism.
(n) Allow the holding and travel mechanism to perform the scheduled activity.
(o) Stop and Wait a Dry Time
(p) Program the laser cutter mechanism according to paper's desired dimensions.
(q) Allow the laser cutter mechanism to execute the programmed paper's cuts
(r) Remove pieces of papers

Alternatively, between steps (o) and (p) turn over the parent sheet of paper and repeat step (n) to apply the blend composition to the other surface of the parent sheet of paper, to ensure there is applied on both sides

1. A composition consisting of at least one previously selected and extracted cannabinoid and at least one previously selected and extracted terpene to be applied on a material in order to be absorbed and then released by heat for recreational or medical purposes, and wherein for each one liter of composition are between 2 to 200 gr of cannabinoid, and between 5 to 100 ml of terpene.

2. The composition of claim 1, wherein said composition can be applied by impregnate to a paper to made any of following products; rolling paper, pre rolls, cones

3. The composition of claim 1, wherein that can be applied to made any of following products;
vaporizer, body oil, dermal cream, beverage drink, chewing gum, scent bar, bath soap, air freshener

4. The composition of claim 1, wherein the cannabinoid is selected from; cannabidiol, tetrahydrocannabinol, Cannabinol, Delta-8-tetrahydrocannabinol, Delta-9-Tetrahydrocannabinol, Delta-10 tetrahydrocannabinol, Tetrahydrocannabiphorol, and Hexahydrocannabinol

5. The composition according to claim 1, consisting of a cannabidiol and at least one antinociceptive terpene for treatment of pain

6. The composition according to claim 1, consisting of a cannabidiol and at least one terpene selected from; D-limonene, Eugenol, Linalool, Geraniol for activates calming effects for states of anxiety

7. The composition according to claim 1, consisting of a cannabidiol and at least one terpene selected from; Linalool, β-caryophyllene, D-limonene, Humulene for activates calming effects for states of insomnia

8. The composition according to claim 1, consisting of a cannabidiol and at least one terpene selected from; α-pinene, β-pinene, Borneol, Linalool, D-Limonene, Geraniol that activates therapeutic effects for increase sex drive

9. The composition according to claim 1, consisting of a cannabidiol and at least one terpene selected from; Citronellol, Geraniol, Linalool, limonene, α-pinene, Sabinene, for activates therapeutic effects for individual relaxation, generating a state of well-being and relaxation in the short term

10. The composition according to claim 1, consisting of a cannabidiol and at least one terpene selected from; Limonene, Linalyl acetate, Linalool, α-pinene, Borneol, that activates therapeutic effects for group's relaxation, generating a state of well-being and relaxation in the long term

11. The composition according to claim 1, consisting of a cannabidiol and at least one terpene selected from; α-pinene, Borneol, Linalyl acetate, Linalool, β-pinene for activates therapeutic effects for reduce and controlling acute stress disorder

12. The composition according to claim 1, wherein for each one liter of composition are between 52 to 64 ml of terpenes, and between 36 to 49 gr of cannabidiol.

13. A paper wherein its surface is covered by a layer adhered by spraying of a mixture composition of at least one previously selected and extracted cannabinoid and at least one previously selected and extracted terpene, and wherein for each one liter of composition are between 2 to 200 gr of cannabinoid, and between 5 to 100 ml of terpene.

14. The paper of claim 13, wherein the cannabinoid is selected from; CBD isolate, CBD full spectrum

15. The paper of claim 14, wherein the composition consisting of at least 60% purity CBD

16. The paper of claim 15, wherein the composition consisting of at least 81% purity CBD

17. The paper of claim 16, wherein the composition consisting of at least between 90% to 99.9% CBD purity

18. The paper of claim 13, wherein is vegetal origin selected from; linen, wood, rice or hemp

19. A process to apply a composition of at least one previously selected and extracted cannabinoid and at least one previously selected and extracted terpene and wherein for each one liter of composition are between 2 to 200 gr of cannabinoid, and between 5 to 100 ml of terpene, on a paper surface with the corresponding steps as follow:
(a) Supply a paper mother sheet obtained from vegetable, selected from; linen, wood, rice or hemp.
(b) Supply a flat surface with dimensions slightly larger than said paper mother sheet.
(c) Fix paper mother sheet corners to said flat surface.
(d) Supply a mix composition of CBD with at least one terpene.
(e) Supply a container that can be sealed in which to store the previous mix composition
(f) Supply spraying medium to apply the previous mixture on the surface of the paper mother sheet, where said spray means are located in superior proximity with respect to said sheet of paper and can be connected by sealed union with said container
(g) Supply a holding and travel mechanism to hold the spraying medium, wherein said mechanism allows an automated two-dimensional path of said spraying medium, parallel and over proximity to paper mother sheet surface.
(h) Supply a programmable laser cutter mechanism.
(i) Prepare CBD and at least one terpene mix composition.
(j) Pour a portion of said mix composition into the container and seal.
(k) Connect the container with the spraying medium.
(l) Hold the spraying medium with the holding and travel mechanism.
(m) Program the holding and travel mechanism.
(n) Allow the holding and travel mechanism to perform the scheduled activity.
(o) Stop and wait a dry time
(p) Program the laser cutter mechanism according to paper's desired dimensions.
(q) Allow the laser cutter mechanism to execute the programmed pieces paper's cuts.
(r) Remove pieces of papers.

20. The process of claim 19, wherein between steps (o) and (p) turn over the parent sheet of paper and repeat step (n) to apply the blend composition to the other surface of the parent sheet of paper, to ensure there is applied on both sides.
